(19) United States
(12) Patent Application Publication
(10) Pub. No.: US 2011/0082403 A1
(43) Pub. Date: Apr. 7, 2011

(54) RECOVERY FOOT AND ANKLE BRACE

(76) Inventor: Melissa Hill, Charlotte, NC (US)

(21) Appl. No.: 12/573,962

(22) Filed: Oct. 6, 2009

(51) Int. Cl. A61F 5/00

(52) U.S. Cl. 602/28

(57) ABSTRACT

A foot and ankle recover brace consisting of an elastic sock body having graduated compression, a strap that is attached at its center to the lower portion of the sock body proximate to the metatarsal heads and having a right side and a left side that continue upward along the medial and lateral sides of the foot. A Velcro loop patch is also attached to the lower portion of the elastic sock and each end of the strap contains a Velcro hook patch. The straps are inserted into attachment loops located on the calf portion of the sock, one attachment loop located on the left side of the calf and another attachment loop located on the right side of the calf. Once the straps are inserted into the attachment loops they are pulled downward and attached to the Velcro loop patch attached to the lower portion of the sock and adjusted according to the desired amount of flexation. The sock also contains at least one pocket located on the bottom portion, the top portion, the heel portion or the calf portion of the sock to house a hot or cold gel pack, if desired.

### RELATED APPLICATIONS

There are no applications related to this application.

### FIELD OF THE INVENTION

The present disclosure and related invention relates to a brace for aiding in the treatment and recovery of many foot and ankle injuries. More specifically, the invention relates to an elastic graduated compression sock with adjustable straps and reusable gel packs designed to aid in the recovery of foot and ankle injuries.

### BACKGROUND OF THE INVENTION

The foot and ankle alone contain 26 bones or one quarter of the bones in the human body. There are more than 100 muscles, tendons and ligaments and a network of blood vessels, nerves, skin and soft tissue contained in the foot and ankle. Therefore, it is not surprising that millions of people each year are faced with foot, ankle or lower leg pain and injury. Injuries such as plantar fasciitis, Achilles tendonitis, shin splints, peroneal tendonitis and posterior tibial tendonitis can be caused from being overweight, from excessive standing, walking, running or general overuse, from consistently wearing high heels, or from sports or other injuries.

Symptoms of these and other foot, ankle and lower leg injuries include chronic pain, swelling, tenderness, redness, and varying degrees of restricted movement. Treatment can take many forms including drugs, orthotics, injections and surgery. Typical non-surgical and non-pharmacological treatments for the above-named injuries include rest, compression of the affected area, stretching and/or cold therapy treatment. Currently, there are a variety of products on the market intended as home use remedies that attempt to alleviate pain and facilitate recovery.

There are several types of night splints on the market that consist of hard plastic shells that are very bulky causing limited mobility and difficulty sleeping. There are also several therapeutic compression or support socks available to aid in support, circulation and athletic performance. Alternatively, there are restraint/support devices that aim to hold the foot in a neutral to slight dorsiflection and limit the plantar flexion of the ankle by using straps positioned around the leg and foot.

However, these products have produced somewhat limited benefits. While the compression sock aids in support and circulation, restraint devices help to stretch certain areas of the foot or leg, and cold therapy can be achieved by using a cold compress, there is a need in the field for a novel device that can aid in the recovery of foot, ankle and lower leg injuries by combining these remedies in a single foot and ankle support and recovery device.

### SUMMARY OF THE INVENTION

The foot and ankle brace of the present disclosure and related inventions includes an elastic graduated compression knee high sock having two adjustable straps, and several strategically located pockets for the insertion of reusable gel packs. The bottom of the sock contains a reinforced strap running from medial to lateral directly under the metatarsal heads. The two side straps are sewn into this strap on the plantar surface and run distal to proximal and attach to two plastic rings located on the sock at approximately mid-calf. The side straps contain a Velcro strip that allows for adjustment of the brace. Several pockets are stitched into the sock to hold reusable gel packs. The pockets are located on the plantar surface (along the plantar fascia), posteriorly (over the Achilles tendon) and laterally (over the peroneal tendon).

### DESCRIPTION OF THE DRAWINGS

FIG. 1 is a perspective view of the foot and ankle brace of the present invention with unattached straps.

FIG. 2 is a perspective view of the foot and ankle brace of FIG. 1 with attached straps.

FIG. 3 is a perspective view of the underside of the foot and ankle brace of FIG. 1 with attached straps.

FIG. 4 is a side view of the foot and ankle brace of FIG. 1 with attached straps.

FIG. 5 is a front view of the foot and ankle brace of FIG. 1 with attached straps.

### DETAILED DESCRIPTION OF PREFERRED AND ALTERNATE EMBODIMENTS

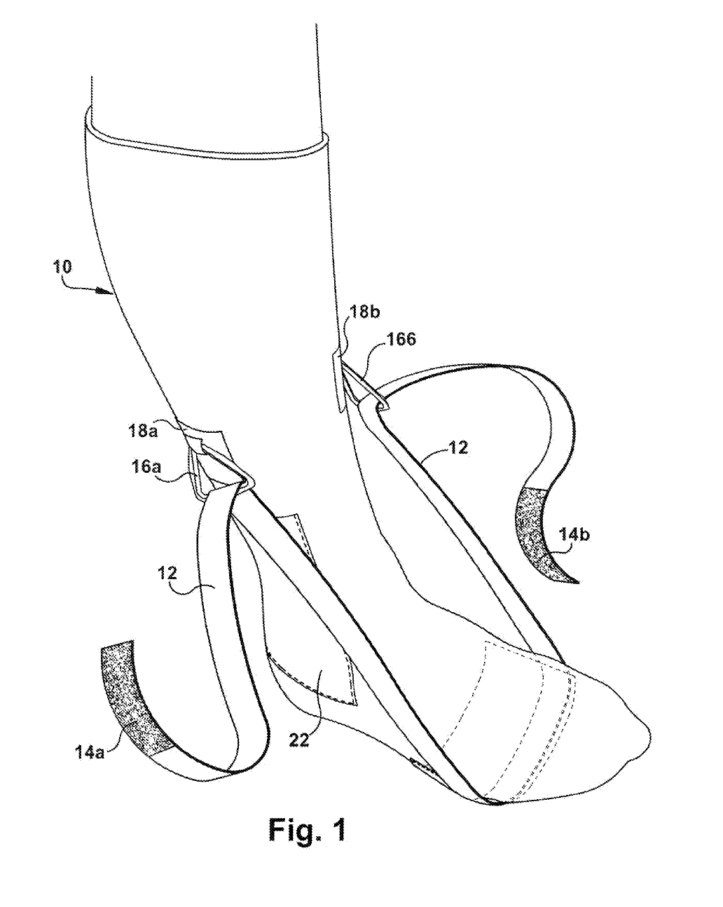

The foot and ankle brace of the present disclosure and related inventions (hereinafter referred to as the “brace”) covers the foot and ankle and extends upward to just below the knee, as shown in FIG. 1. In a preferred embodiment, the body of the brace 10 is made from an elastomeric material that contains graduated compression which varies from a maximum at the foot region of the brace to a minimum at the top of the calf. The compression may range, in one embodiment, from 15 to 30 mmHg, from 15 to 20 mmHg in another embodiment, and preferably from 25 to 30 mmHg. The compression gradient can be formed in any manner known in the art, such as varying the tension of the yard during manufacture or using yarns of different elasticity. The graduated compressive nature of the brace has been shown to improve the return of blood to the heart via the inferior and superior vena cava and the coronary sinus, which is also known as venous return. Other benefits include, but are not limited to: decreasing edema or swelling caused by fluid in body tissues that usually occurs in the foot, ankle and legs; and decreasing lactic acid, a water-soluble liquid produced in muscles as a result of anaerobic glucose metabolism, which is thought to cause muscle soreness and fatigue during exercise.

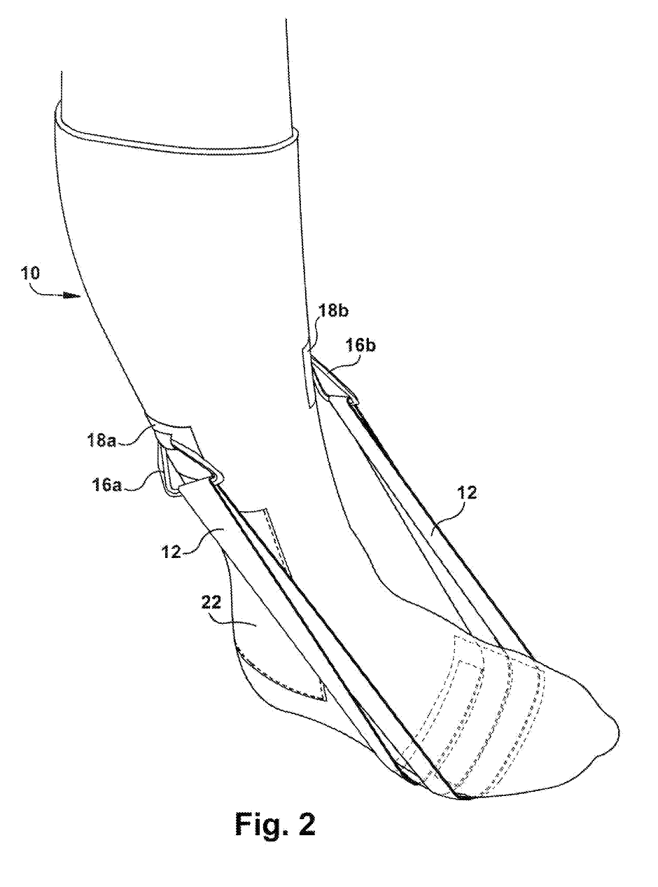

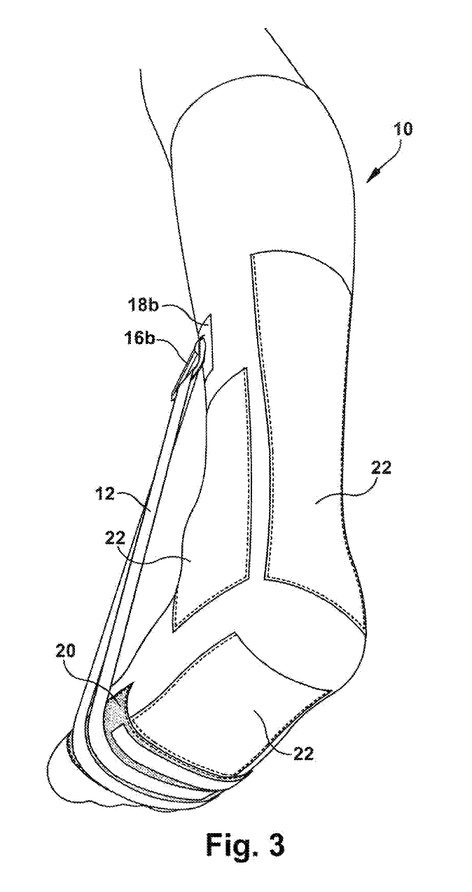

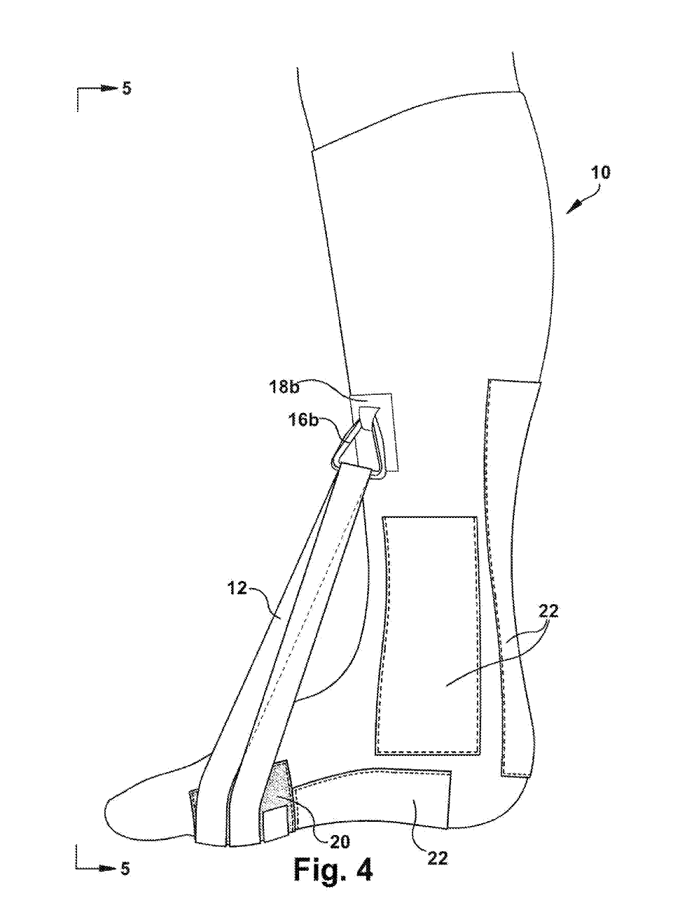

A reinforced Velcro loop patch 20 is sewn or otherwise attached to the bottom of the brace 10 and runs from medial to lateral directly under the metatarsal heads, as shown in FIG. 3. In a preferred embodiment, the patch 20 is approximately two inches long and approximately 3.5 to 4 inches wide and contains Velcro loops on the outer, downward facing surface. A single strap 12 is sewn or otherwise attached to the reinforced Velcro patch 20 and each end of the strap 12 extends upward and parallel along opposing sides of the brace 10, creating a left and right side strap. In the preferred embodiment, each side strap extends upward for approximately 14 to 18 inches. A strip of Velcro hooks 14a, 14b is located on each distal end of the side straps 12. As shown in FIG. 4, a piece of reinforced, non-elastic material 18a, 18b is located at both the right and left side of the brace 10 at approximately mid-calf lever, slightly anterior to the medial and lateral aspects of the leg. Attached to this material is a ring 16a, 16b through which each side strap 12 is inserted. The rings 16a, 16b are preferably made of plastic, but can be made of metal or any other suitable material. As shown in FIG. 2, once each strap 12 is inserted into the ring 16a, 16b, the straps 12 can be then folded downward and the strip of Velcro hooks 14a, 14b attached to each side strap 12 can be attached to the reinforced patch of Velcro loops 20 located at the bottom of the brace 10 directly under the metatarsal heads.

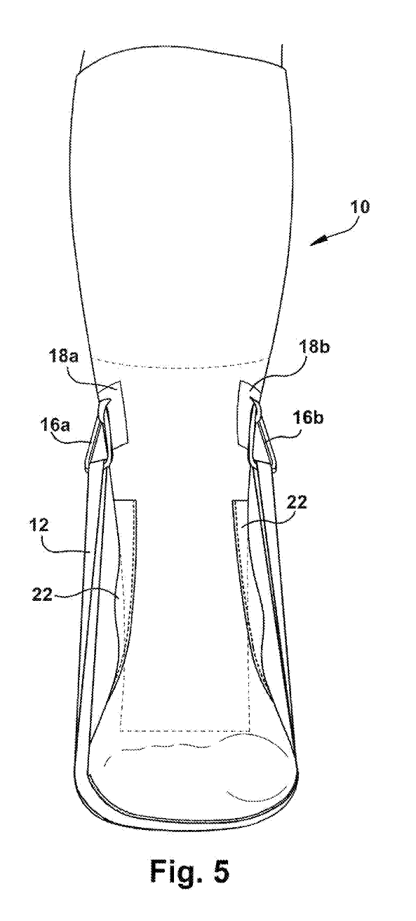

There are several pockets 22 strategically attached to the brace 10 to house gel packs (not shown). The pockets 22 are located on the plantar surface (along the plantar fascia and shown in FIGS. 3 and 5), posteriorly (over the Achilles tendon and shown in FIGS. 3 and 4) and laterally (over the peroneal tendon and shown in FIG. 4). The gel packs may be used for either cold or heat therapy. The gel packs may be placed in the freezer until frozen and then placed into one or more of the pockets contained on the brace. The gel pack would conform to the affected area and help relieve pain and inflammation. The gel packs may also be heated in the microwave and placed into one or more pockets to help warm the muscles prior to stretching to get the maximal physiologic stretch. Alternately, ice packs or any other suitable cooling or heating device may be placed within the pockets 22 to apply either cold or heat therapy.

Once the brace 10 has been placed onto the foot, the straps 12 can be adjusted as necessary. The straps 12 are pulled through the attached rings 16a, 16b and folded downward so that the distal end of each strap containing the strip of Velcro hooks 14a, 14b can be attached to the Velcro loop patch 20 located at the bottom of the brace 10. The straps 12 provide an anatomic stretch to the plantar fascia and Achilles tendon. The wearer may use the straps 12 to adjust the angle of ankle joint dorsiflexion. The straps 12 may be loosely attached to the Velcro loop patch 20 for a slight stretch or the straps 12 may be attached more tightly for a deeper stretch. The maximum allowable stretch would be 15-20 degrees of ankle joint dorsiflexion. Positioning the distal aspect of the Velcro loop patch 20 starting at the metatarsal heads, as opposed to the top of the phalanges, protects the metatarsal phalangeal joints from getting unnecessarily stretched.

The brace 10 may be used as a supplemental home remedy for many foot, ankle or lower leg injuries. One of the most common causes of foot or heel pain is plantar fasciitis. Plantar fasciitis is an inflammation of the plantar fascia, which is the thick connective tissue that spans between the medial calcaneal tubercle and the proximal phalanges of the toes. Another common cause of foot pain is Achilles tendonitis which is an irritation and inflammation of the Achilles tendon. The Achilles tendon is the large tendon at the back of the ankle that connects the large calf muscles to the heal bone. These and many other foot, ankle or lower leg injuries can be supplementally treated using the brace of the present disclosure and related inventions. The brace 10 keeps pressure on the plantar fascia so that it heals in a stretched position over a period of time by not allowing the plantar fascia to contract or be in a position of plantar flexion while in a prone position. The brace may be comfortably worn at night because it has a thin, soft outer surface and provides no significant restraint of the leg.

The brace of the present disclosure and related invention may also be used by athletes or others as a preventative measure. Regular stretching of the shins is one key to avoiding shin splints, which is a common overuse injury for runners or aggressive walkers. The stretching also strengthens the Achilles tendon and the medial and lateral foot. One of the side straps 12 may be left unconnected to allow stretching of either the medial or lateral foot. Consistent use of the brace 10 may also help strengthen the arch of the foot which is very often prone to injury. The brace 10 may also be worn by athletes after activity to improve circulation or to apply heat or cold therapy to any inflammation occurring in the foot or calf area.

The foregoing embodiments of the present invention have been presented for the purposes of illustration and description. These descriptions and embodiments are not intended to be exhaustive or to limit the invention to the precise form disclosed, and obviously many modifications and variations are possible in light of the above disclosure. The embodiments were chosen and described in order to best explain the principle of the invention and its practical applications to thereby enable others skilled in the art to best utilize the invention in its various embodiments and with various modifications as are suited to the particular use contemplated. It is intended that the invention be defined by the following claims.

I claim:

1. A foot and ankle recovery brace comprising:
an elastic sock with graduated compression having an upper portion that extends over the top of the foot and top of the toes, a lower portion that extends over the sole of the foot and bottom of the toes, a heel portion that extends over the heel, a calf portion that extends over the calves and an open end positionable proximate to the upper calf;
a Velcro loop patch attached to the lower portion of the elastic sock that runs from medial to lateral directly under the metatarsal heads;
a strap attached to the Velcro loop patch that continues upward along both the medial and lateral sides of the foot and contains a Velcro hook strip at each end of the strap;
two attachment rings attached to the calf portion of the elastic sock, one on the right side and one on the left side at approximately mid-calf level and slightly anterior to the medial and lateral aspects of the leg;
at least one pocket attached to the elastic sock; and
at least one gel pack contained inside the at least one pocket,
wherein the strap is positioned through the two attachment rings, folded downward and attached to the Velcro loop patch.

2. The foot and ankle recovery brace of claim 1, wherein the attachment ring is attached to a non-elastic reinforcement material that is attached to the calf portion of the elastic sock.

3. The foot and ankle recovery brace of claim 1, wherein the graduated compression is in the range of 25 to 30 mmHg.

4. The foot and ankle recovery brace of claim 1, wherein the graduated compression is in the range of 15 to 30 mmHg.

5. The foot and ankle recovery brace of claim 1, wherein the graduated compression is in the range of 15 to 20 mmHg.

6. The foot and ankle recovery brace of claim 1, wherein the maximum allowable stretch is 15 to 20 degrees of ankle joint dorsiflexion.

7. The foot and ankle recovery brace of claim 1, wherein the at least one pocket is located on the plantar surface.

8. The foot and ankle recovery brace of claim 1, wherein the at least one pocket is located over the Achilles tendon.

9. The foot and ankle recovery brace of claim 1, wherein the at least one pocket is located over the tibialis posterior tendon.

10. The foot and ankle recovery brace of claim 1, wherein the pocket is located over the peroneal tendon.

11. The foot and ankle recovery brace of claim 1, wherein four pockets are attached to the elastic sock, one pocket located on the plantar surface, one pocket located over the Achilles tendon, one pocket located over the tibialis posterior tendon, and one pocket located over the peroneal tendon.

12. The foot and ankle recovery brace of claim 11 further comprising four gel packs, each gel pack located within each of the four pockets attached to the elastic sock.

13. A foot and ankle recovery brace comprising:
an elastic sock body with graduated compression in the range of 15 to 30 mmHg, having an upper portion that extends over the top of the foot and toes, a lower portion that extends over the sole of the foot and bottom of the toes, a heel portion that extends over the heel, a calf portion that extends over the calf and an open end positionable proximate to the upper calf;
a Velcro loop patch attached to the lower portion of the elastic sock that runs from medial to lateral directly under the metatarsal heads;
a strap attached to the Velcro loop patch that continues upward along both the medial and lateral sides of the foot and contains a Velcro hook strip at each end of the strap;
two pieces of reinforced, non-elastic fabric attached to the calf portion of the elastic sock, one on the right side and one on the left side, at approximately mid-calf level and slightly anterior to the medial and lateral aspects of the leg;
two attachment rings attached to the two pieces of reinforced, non-elastic fabric;
at least four pockets attached to the elastic sock, one pocket located on the plantar surface, one pocket located over the Achilles tendon, one pocket located over the tibialis posterior tendon, and one pocket located over the peroneal tendon; and
at least four gel packs, one gel pack contained inside each of the four pockets,
wherein the strap is positioned through the each attachment ring, folded downward and attached to the Velcro loop patch.

14. The foot and ankle recovery brace of claim 13, wherein the maximum allowable stretch is 15 to 20 degrees of ankle joint dorsiflexion.
